(19) United States
(12) Patent Application Publication
(10) Pub. No.: US 2017/0224349 A1
(43) Pub. Date: Aug. 10, 2017

(54) VASCULAR COMPRESSION ASSIST DEVICE AND METHOD OF TACTILE HEMOSTASIS

(71) Applicant: David SCHNEIDER, Orlando, FL (US)

(72) Inventor: David SCHNEIDER, Orlando, FL (US)

(21) Appl. No.: 15/425,248

(22) Filed: Feb. 6, 2017

Related U.S. Application Data
(60) Provisional application No. 62/291,113, filed on Feb. 4, 2016.

(51) Int. Cl. A61B 17/12; A61H 1/00

(52) U.S. Cl. CPC A61B 17/12009; A61H 1/008; A61B 17/12; A61B 2017/12004

(57) ABSTRACT

A vascular compression assist device to achieve hemostasis comprises a generally C-shaped self-biasing member having a first free end spaced apart from a second free end forming a gap there between and the first end and second end bias in directions toward one another. An opening is provided through the member adjacent to the first end, and the opening is configured to receive one or more fingertips of a user. The member is preferably composed of a medical grade shape memory plastic material so the member is resilient to open the gap adapting the member to receive a body part within which a surgically punctured blood vessel is disposed and the first end and second end bias toward one another upon release of the device.

### CROSS REFERENCE TO RELATED APPLICATIONS

This application claims priority benefit to U.S. Provisional Patent Application Ser. No. 62/291,113, entitled “A Vascular Compression Assist and Method of Applying Tactile Pressure to Achieve Hemostasis” filed Feb. 4, 2016.

### FIELD OF THE INVENTION

This invention relates generally to vascular compression devices, more specifically, to such devices and methods that facilitate tactile hemostasis relative to a blood vessel.

### BACKGROUND OF THE INVENTION

Following any percutaneous catheterization, hemostasis is required to prevent bleeding complications resulting from removing the catheter. Currently, manual pressure and venous closure devices are widely used. Although venous closure devices have gained popularity, the gold standard for removing a catheter is with manual pressure. A few devices exist to assist manual pressure or take complete control over hemostasis with mechanical pressure.

The femoral artery is a high pressure vessel which generally requires direct pressure to achieve hemo stasis following a direct catheterization. If pressure is not provided on the femoral vessel following sheath removal, severe complications could follow. A few complications of catheter removal include, but are not limited to: hypovolemia, bradycardia, hypovolemic shock, hypotension, ischemia, and death. However, hemostasis can be established with direct pressure held on the vessel proximal to an insertion site. Direct pressure slows blood flow in the vessel, allowing for a clot to form. This process for achieving hemostasis traditionally takes 20-30 minutes. This can result in significant fatigue, pain or discomfort in the fingers, wrist, and forearm of the individual applying pressure. Prolonged or repeated performance of this process can result in work-related musculoskeletal disorders.

Various types of manual compression solutions have been developed to address these issues. Current handheld models include the ComfortPress™, EZ Hold™, and D-Stat™. These devices all are comprised of a handle, a shaft extending downward into a base that comes into direct contact with the patient. These aforementioned manual hemostatic compression devices are far from ideal. All these devices provide for straight vertical non-tactile compression at a single location; however, these devices fail to account for any tactile hemostasis. Tactile hemostasis allows for the femoral pulse to be palpated, hematomas to be felt, and pressure to be adjusted slightly due to feel.

### SUMMARY OF THE INVENTION

The invention provides for manual and tactile manual and tactile vascular compression device for assisting a user to control bleeding and achieving hemostasis. The invention assists the user in controlling bleeding and achieving hemostasis with a hand held device that provides manual and tactile compression of a vascular puncture site at both the skin level and blood vessel level simultaneously.

A further object of the invention is to assist a user in controlling bleeding and achieving hemostasis after removing a catheter or cannula from a blood vessel by providing manual tactile compression along the vessel.

A further object of the invention is to provide for a manual tactile compression device that facilitates hemostasis following percutaneous catheterization via artery or vein.

A further object of the device is to reduce the user's personal exertion and grip force when achieving manual tactile hemostasis.

An embodiment of the invention may include a vascular compression device that has a C-shaped body with an opening at one or both ends for receiving fingers a thumb of a user, enabling a user to apply tactile pressure to a vein, vessel or artery. In a preferred embodiment, the device is one unitary piece. The device is designed to have a pressure bias generating from the center of the device which forces the device to close on itself.

The C-shape of the device fits to the contour of the user's hand. The device, unlike any prior art, allows for mechanical and manual tactile vascular compression to work together. Another preferred embodiment would include the device being sterile. In a most preferred embodiment, the device is used only once and is disposable.

Key differences between the present invention and prior art devices include:
1) more direct control of hemostasis;
2) tactile hemostasis;
3) user is engaged with tactile hemostasis for 100% of the time used to achieve hemostasis;
4) the present invention is C shaped with perforations for fingers and thumb;
5) the present invention delivers aided mechanical pressure from the C shaped design; and,
6) the present invention combines mechanical and manual tactile pressure for optimal hemostasis.

Embodiments for a vascular compression assist device to achieve hemostasis may comprise a first arm having an opening adapted for receiving one or more finger tips of a user and the first arm has a free end adjacent to the opening; and, a second arm having a free end wherein the ends of the first arm and second arm are spaced apart forming a gap there between. A curved mid-section is integrally formed with the first arm and second arm, wherein the device has a generally C-shaped configuration having the gap between the first arm and second arm for receiving a body part in which a blood vessel is disposed, and the first arm, second arm and mid-section are composed of a same shape memory material such that the first arm and second arm are self-biasing toward one another.

The gap has a dimension from the end of the first arm to a point associated with the second arm and the dimension of the gap is less than a width dimension of a body part to be received between first arm and second arm, and the first arm and second arm bias toward one another to apply pressure to the blood vessel to achieve hemostasis associated with the blood vessel.

In an aspect of the invention, the device may comprise a window, at least a portion of which is disposed on the first arm, and the opening of the first arm is between the window and the free end of the first arm.

In another aspect of the invention, the opening of the first arm is a first opening of the device and the device further comprises a second opening in the second arm and the second opening is configured to receive at least a portion of a thumb of the user.

Embodiments of the invention may also include a method of tactile hemostasis of a blood vessel at a surgical insertion site of the blood vessel, and the method comprises:
providing a self-biasing C-shaped device having a first arm spaced apart from a second arm forming a gap there between for receiving a body part in which the blood vessel is disposed, and the first arm includes an opening configured to receive one or more fingertips of a user of the device;
manually gripping the device and opening the gap to receive the body part;
positioning an end of the first arm proximal the blood vessel;
applying pressure to the blood vessel by allowing the first arm and second arm to bias toward one another;
inserting one or more fingertips through the opening;
applying pressure against the body part at the blood vessel via the fingertips through the opening to achieve and detect hemostasis of the blood vessel.

In accordance with the above method, the device is preferably composed of a shape memory material such that the first arm and second arm bias toward one another after the gap is opened and an outward force applied to the first arm and second arm is released.

### DESCRIPTION OF THE DRAWINGS

The features and advantages of the present invention will become apparent from the following detailed description of the invention when read with the accompanying drawings.

FIG. 1 is a perspective view of a vascular compression assist device in accordance with aspects of the invention.

FIG. 2 is a side elevational view of the device.

FIG. 3 is a cross-sectional view of the device taken along line 3-3 of FIG. 2.

FIG. 4 is a side view of the device with a user's fingers and thumbs inserted and ready for use.

FIG. 5 is a perspective view of the assist being used to apply tactile pressure to a blood vessel, vein or artery of a patient.

FIG. 6 is a sectional view of the application of the assist to the patient of FIG. 5.

FIG. 7 is a rear perspective view of a second embodiment of the invention for a vascular compression assist device in accordance with aspects of the invention.

FIG. 8 is front perspective view of the device.

FIG. 9 is a side elevational view of the device.

FIG. 10 is a front elevational view of the device.

FIG. 11 is a top view of the device.

FIG. 12 is a cross-sectional view of the device take along line 10-10 of FIG. 8.

FIG. 13 is a perspective view of the assist device used to apply tactile pressure to a blood vessel, vein or artery of a patient.

FIG. 14 is a flowchart including steps of a method of tactile hemostasis in accordance with aspects of the invention.

### DESCRIPTION OF THE INVENTION

Embodiments are described herein with reference to the attached figures wherein like reference numerals are used throughout the figures to designate similar or equivalent elements. The figures are not drawn to scale and they are provided merely to illustrate aspects disclosed herein. Several disclosed aspects are described below with reference to non-limiting example applications for illustration. It should be understood that numerous specific details, relationships, and methods are set forth to provide a full understanding of the embodiments disclosed herein. One having ordinary skill in the relevant art, however, will readily recognize that the disclosed embodiments can be practiced without one or more of the specific details or with other methods. In other instances, well-known structures or operations are not shown in detail to avoid obscuring aspects disclosed herein. The embodiments are not limited by the illustrated ordering of acts or events, as some acts may occur in different orders and/or concurrently with other acts or events. Furthermore, not all illustrated acts or events are required to implement a methodology in accordance with the embodiments.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

A vascular compression assist apparatus and method for applying tactile manual pressure onto an area of a patient generally includes a vessel used for puncture or cannulation during a procedure for the purpose of achieving hemostasis. The vascular compression assist device is comprised of a single structure and body that is generally C-shaped in nature. The C-shaped body has two perforated areas at either end. The perforated areas allow the user's fingers or thumb to pass through for tactile contact with the patient. In use the device and perforated areas are positioned proximal to the insertion site. In use, the device will be stretched open and placed proximal of the patient's insertion site with tactile feel placed proximally on the vessel. In use the exertional pressure on the device will be relaxed with tactile pressure on the vessel creating manual and mechanical tactile pressure proximal of the insertion site. After the catheter is removed, the device allows the user to mitigate and exert more manual and mechanical tactile pressure proximal to the insertion site. The device is utilized in aiding in manual and mechanical tactile pressure until hemostasis is achieved.

While embodiments disclosed herein may be described in relation to achieving hemostasis of a catheterized posterior femoral artery, the invention is not so limited. For example, the device may be dimensioned to be functional in connection with achieving hemostasis in blood vessels associated with different body parts. To that end, the invention is not limited to achieving hemostasis in bloody vessels of human body parts and may be used in procedures with other mammals. In addition, the term blood vessel as used herein shall mean a tubular structure carrying blood through tissues and organs, and may include a vein, artery or capillary.

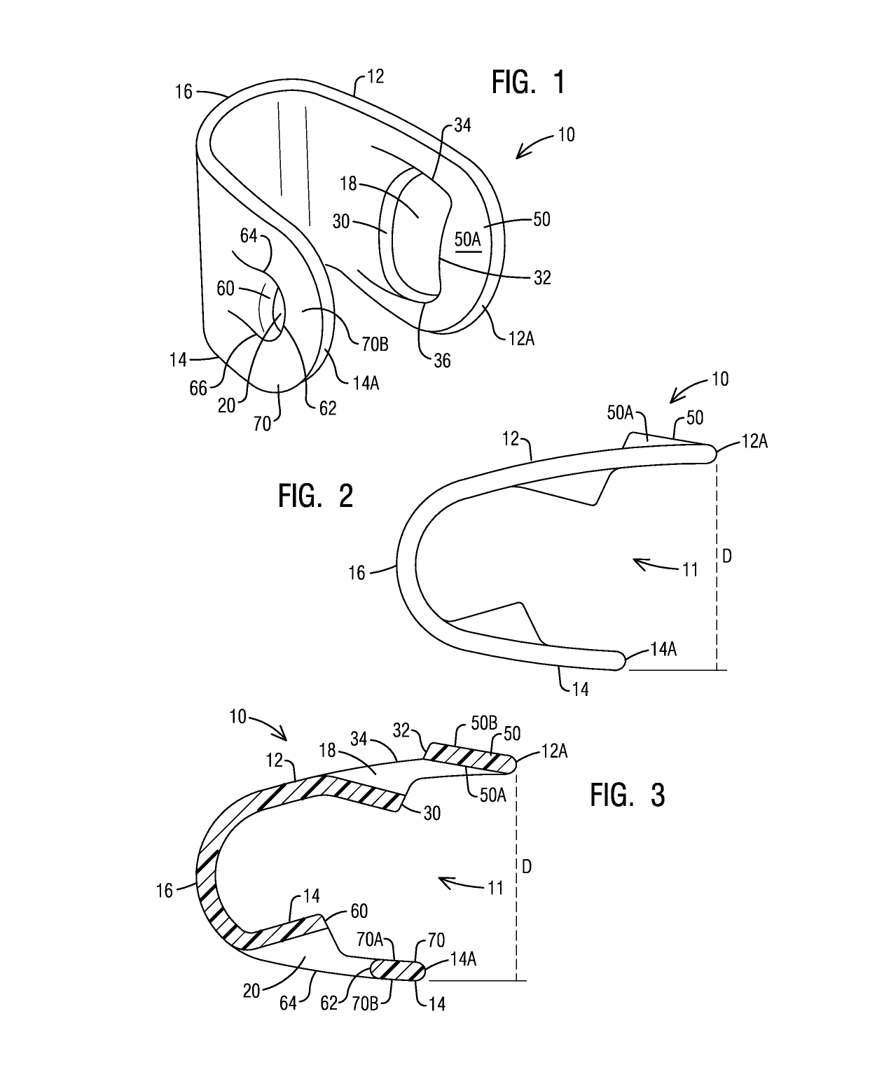

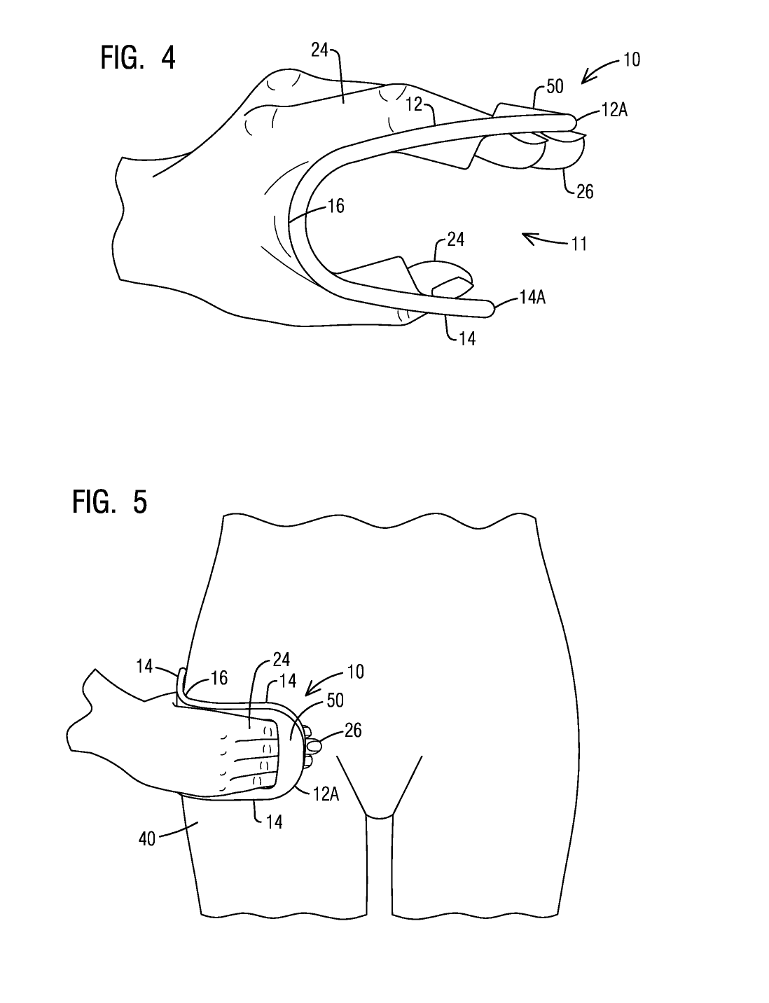

With respect to FIG. 1-4, the vascular compression assist 10 shown is generally C-shaped including a first arm 12 and a second arm 14 that is spaced apart from the first arm 12. The two arms 12, 14 are integrally formed and contiguous with a curved mid-section 16. The first arm includes a free end 12A spaced apart from a freed end 14A of the second arm forming a gap 11 between the first arm 12 and second arm 14 or between the respective free ends 12A, 14A.

The assist 10 is preferably fabricated from an inexpensive shape memory medical grade plastic. Examples of materials may include polypropylene, polyurethane polyethylene, polyvinyl chloride or nylon. In a preferred embodiment, the medical grade plastic material is polypropylene. In as much as the device 10 is fabricated from a shape memory material in which its parts integral to each other as a contiguous single unit, the first and second arms 12, 14 are “self-biasing” toward one another. The term “self-biasing” as used herein means that the device or member is composed of such a shape memory material such that the free ends of the device, or the opposing arms of the device, tend to bias toward one another without the exertion of an external force.

In an embodiment, each arm 12, 14 includes a perforation or opening 18, 20 through which one may insert a thumb 22, fingers 24 or fingertips 26 for gripping and using the assist 10. The opening 18 on the first arm 12 is configured to receive the fingers 24 and/or finger tips 26 of a user; and the opening 20 on the second arm 14 is configured to receive the thumb 22 of a user. However, the invention does necessarily have to include both openings and may include only the opening 18, so that a user may be able to apply tactile pressure to the blood vessel using his/her fingertips 26 to achieve and detect hemostasis in the blood vessel.

As shown in FIGS. 1 and 3, the opening 18 is defined by a plurality of contiguous edges include first and second parallel side edges 30 (lower), 32 (upper) and two opposing end edges 34, 36 forming a generally ovular or rectangular opening 30. The two end edges 44, 46 extend radially outward and upward from the first side edge 30 relative to a curvature of the device 10, and terminate at the upper side edge 32, which is spaced above the lower or first side edge 30. A planar finger engagement member 50 is disposed between the upper edge 32 of opening 18 and the free end 14A of the first arm 14, having an inner surface 50A, and an outer surface 50B. In this manner, a user is able flex an anterior side of his/her fingertips 26 against the engagement member 50 to open the gap 11 as necessary.

The second opening 20 is also defined by a plurality of contiguous edges include first and second parallel side edges 60, 62 and two opposing end edges 64, 66 forming a generally ovular or rectangular opening. The two end edges 64, 66 extend radially inward and upward from the second side edge 62 relative to a curvature of the device 10, and terminate at the first side edge 60, which is spaced above the second side edge 62. A planar thumb engagement member 70 is disposed between the second side edge 52 of opening 20 and the free end 14A of the second arm 14, having an inner surface 70A, and an outer surface 70B. In this manner, a user is able flex an anterior side of thumb 24 against the engagement member 70 to open the gap 11 as necessary.

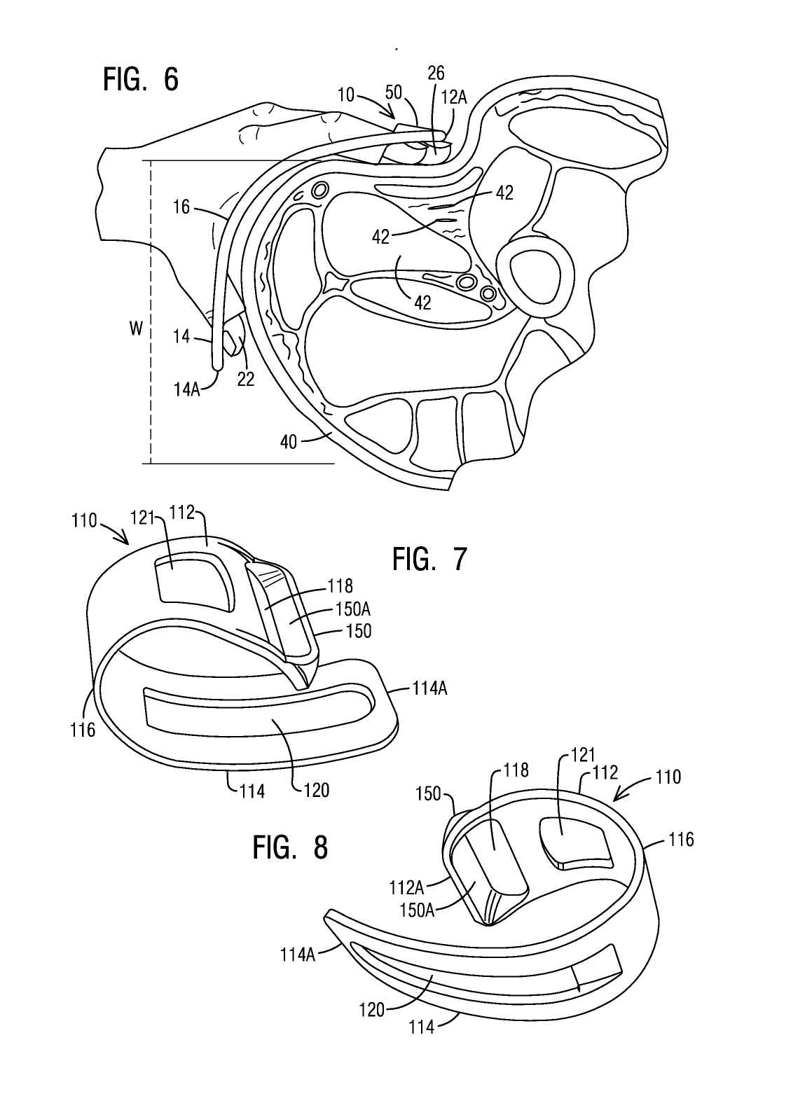

With respect to FIGS. 5 and 6, the device 10 is shown applying pressure to a blood vessel 40 of body part 42. For example, the blood vessel may have an insertion site of a catheter inserted and removed from a femoral artery as a result of a surgical procedure. The gap 11 has a dimension “D” (FIGS. 2 and 3) that is less than a width dimension “W” of a body part 40. Accordingly, a user applies an outward force to the device 10 to open the gap 11 to receive the body part 40 or a portion of body part 40. This may be done by inserting fingers 24 or fingertips 26 in opening 18, and a thumb 22 in opening 20 of the second arm 14, and opening the hank.

When, the free end 12 is positioned proximal the blood vessel 42, the application of force to open the device 10 or gap 11 is released. Upon release, the first and second arms 12, 14 bias towards one another the two arms 12, 14 bias toward another for application of pressure to the blood vessel 42 thereby achieving hemostasis. In an embodiment, the device 10 may be configured to apply from about six pounds to abut ten pounds of pressure to the blood vessel.

The openings 18, 20 allow for application of tactile pressure so one may feel the blood vessel 42 to monitor that state of hemostasis. In an embodiment, the first arm 12 may be longer than the second arm 14, so that the assist 10 may be positioned relative to a blood vessel at an appropriate angle to apply tactile pressure. However, the second arm 14 may be longer than the first arm 12, or the arms 12, 14 may be of equal length. By virtue of the arms 12, 14 biasing toward one another, a user needs only minimal pressure at the blood vessel for purposes of monitoring the state of hemostasis as the assist 10 applies sufficient pressure to achieve hemostasis.

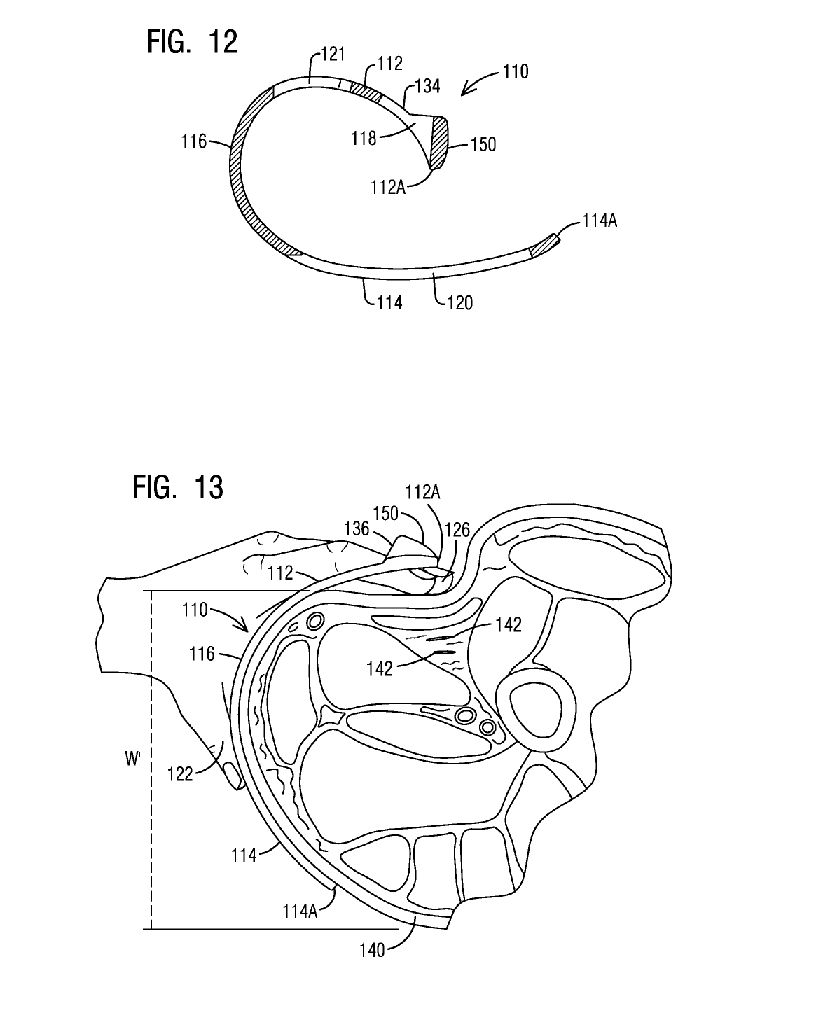

With respect to FIG. 7-13 a second embodiment of the device 110 is shown in which the second arm 114 is longer than the first arm 112. Similar to the above-described embodiment, the device 110 includes a curved mid-section 116 integrally formed and contiguous with the first and second arm 112, 114. The first arm 112 includes a free end 112A spaced apart from a free end a 14A of the second arm 114 forming a gap 111 between the first arm 112 and second arm 114 or between the respective free ends 112A, 114A.

The assist 110 is preferably fabricated from an inexpensive shape memory medical grade plastic. Examples of materials may include polypropylene, polyurethane polyethylene, polyvinyl chloride or nylon. In a preferred embodiment, the medical grade plastic material is polypropylene.

In an embodiment, each arm 112, 114 includes a perforation or opening 118, 120 through which one may insert a thumb 122, fingers 124 or fingertips 126 (FIG. 13) for gripping and using the assist 10. The opening 118 on the first arm 112 is configured to receive the fingers 124 and/or finger tips 126 of a user; and the opening 120 on the second arm 114 is configured to receive the thumb 122 of a user. However, the invention does necessarily have to include both openings and may include only the opening 118, so that a user may be able to apply tactile pressure to the blood vessel using his/her fingertips 126 to achieve and detect hemostasis in the blood vessel.

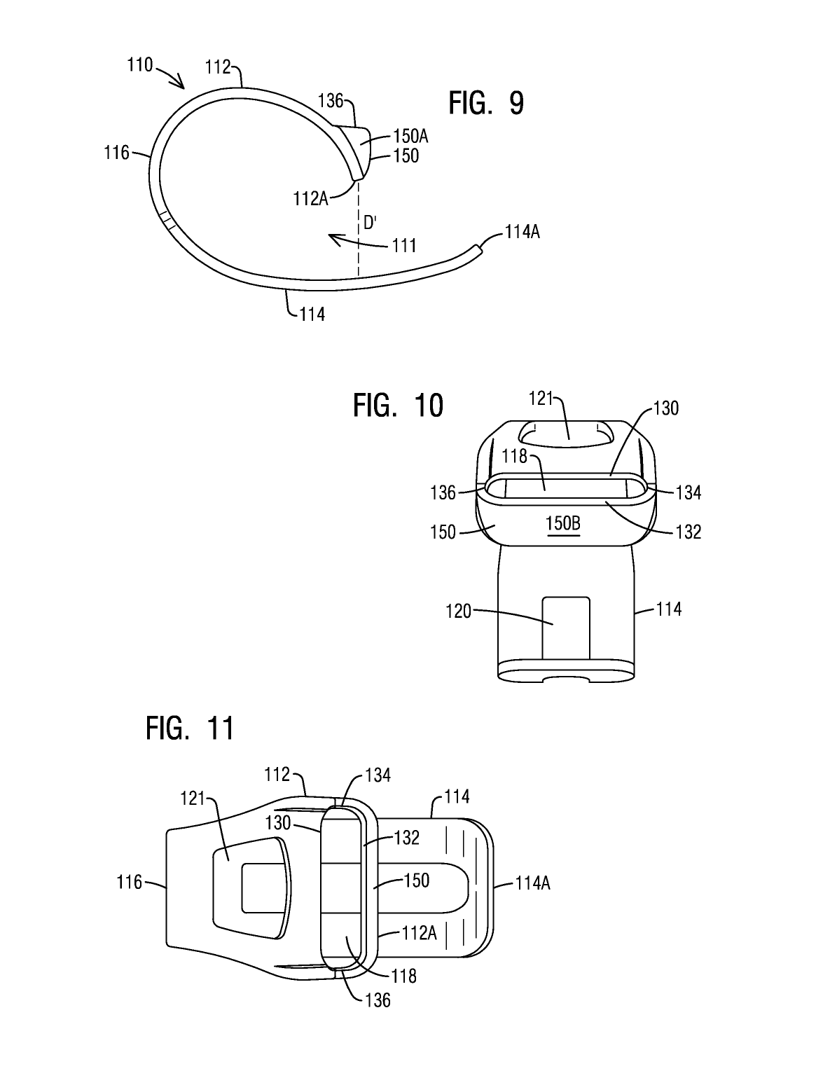

As shown in FIGS. 9, 10 and 11, the opening 118 is defined by a plurality of contiguous edges including first and second parallel side edges 130 (lower), 132 (upper) and two opposing end edges 134, 136 forming a generally ovular a rectangular opening 118. The two end edges 134, 136 extend radially outward and upward relative to a curvature of the device 110, and terminate at the upper edge 132. In reference to FIGS. 7, 8, 9, and 10 a planar finger engagement member 150 is disposed between the upper edge 132 of opening 118 and the free end 14A of the first arm 114, having an inner surface 150A, and an outer surface 150B. In this manner, a user is able flex an anterior side of his/her fingertips 126 to open the gap 111 as necessary.

While the invention is not limited to any particular dimensions, the embodiment shown in has a length dimension of about 5 inches measured from a mid-section 116 point to the free end 114A of the second arm 114. The gap dimension “D′” may be about 3 to 4 inches, preferably about 3.5 inches. In addition, the device has a width dimension of about 2.5 inches at the engagement member 150.

With respect to FIGS. 13, the device 110 is shown applying pressure to a blood vessel 142 of body part 140. For example, the blood vessel may have an insertion site of a catheter inserted and removed from a femoral artery as a result of a surgical procedure. The gap 111 has a dimension “D” that is less than a width dimension “W′” of a body part 140. Accordingly, a user applies an outward force to the device 110 to open the gap 111 to receive the body part 40 or a portion of body part 140. This may be done by inserting fingers 124 or fingertips 126 in opining 118, and a thumb 122 in opening 22 of the second arm 14, and opening the hank.

When, the free end 112A is positioned proximal the blood vessel 142, the application of force to open the device 110 or gap 111 is released. Upon release, the first and second arms 112, 114 bias towards one another the two arms 112, 114 bias toward another for application of pressure to the blood vessel 142 thereby achieving hemostasis. In an embodiment, the device 110 may be configured to apply from about six pounds to abut ten pounds of pressure to the blood vessel.

The opening 118 allows for application of tactile pressure so one may feel the blood vessel 142 to monitor that state of hemostasis. By virtue of the arms 112, 114 biasing toward one another, a user needs only minimal pressure at the blood vessel for purposes of monitoring the state of hemostasis as the assist 110 applies sufficient pressure to achieve hemostasis.

As further shown in FIGS. 7, 8, 10 and 11, a window 121 is provided such that at least a portion of the window 121 is disposed on the first arm 112, and the opening 118 is between the window 121 and the free end 112A. In this manner, a user is able to view an insertion site, for example, to see if external bleeding of a patient occurs to take appropriate remedial measures.

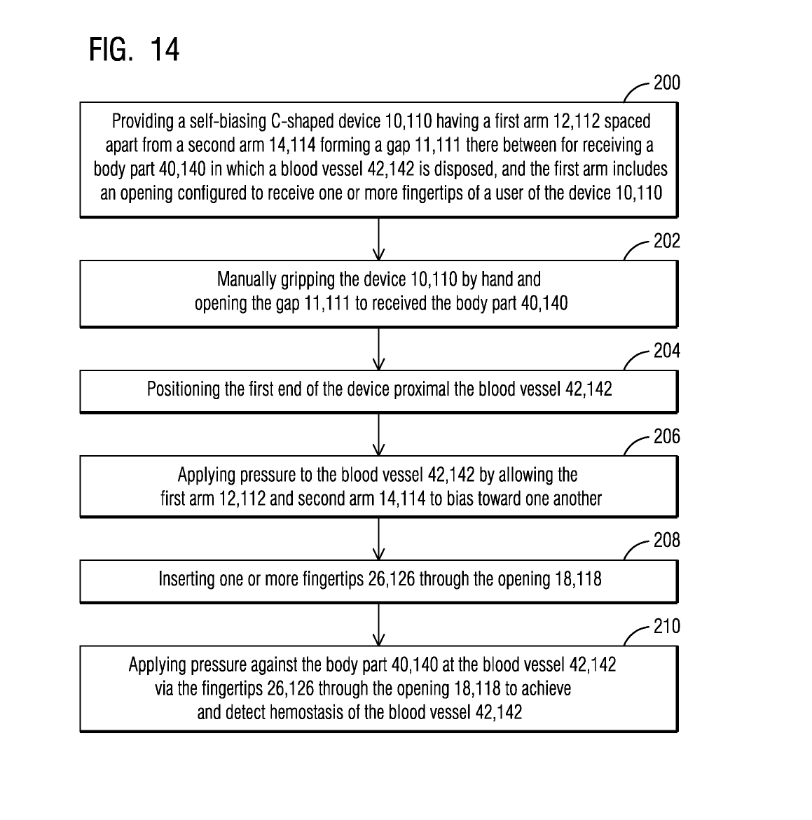

In another aspect of the invention, a method is set forth in the flowchart of FIG. 14. As shown, the method includes step 200 of providing a self-biasing C-shaped device 10, 110 having a first arm 12, 112 spaced apart from a second arm 14, 114 forming a gap 11, 111 there between for receiving a body part 40, 140 in which a blood vessel 42, 142 is disposed, and the first arm includes an opening configured to receive one or fingertips of a user of the device 10, 110.

At step 202 the method includes manually gripping the device 10, 110 by hand and opening the gap 11, 111 to receive the body part 40, 140. The method further comprises at step 204 positioning the first end of the device proximal the blood vessel 42, 142; and, at step 206 applying pressure to the blood vessel 42, 142 by allowing the first arm 12, 112 and second arm 14, 114 to bias toward one another.

The method further comprises at step 208 inserting one or more fingertips 26, 126 through the opening 18, 118. This step 208 may be integrated with step 202, wherein the step 202 of manually gripping the device 10, 110 includes inserting fingertips 26, 126 through the opening 18, 118. Step 210 includes applying pressure against the body part 40, 140 at the blood vessel 42, 142 via the fingertips 26, 126 through the opening 18, 118 to achieve and detect hemostasis of the blood vessel 42, 142.

In an embodiment, the above method is performed in relation to a surgical procedure in which a catheter is inserted into the blood vessel 42, 142 at the insertion site, and the step of positioning the first end 12A, 112A proximal the blood vessel 42, 142 is performed before the catheter is removed from the blood vessel 42, 142 and the step of applying pressure via the fingertips 26, 126 is performed after the catheter is removed.

While the preferred embodiments of the present invention have been shown and described herein, it will be obvious that such embodiments are provided by way of example only. Numerous variations, changes and substitutions will occur to those of skill in the art without departing from the invention herein. Non-limiting examples include a component that is described above as being attached to one part of the apparatus may alternatively be attached to a different part of the apparatus in other embodiments. Parts described as being indirectly connected may be connected directly to each other, and vice versa. Component parts may be assembled from individual pieces or may be integrally formed as a single unit. Alternative types of connectors and alternative materials may be used. The apparatus may be used with other types of power tools. Accordingly, it is intended that the invention be limited only by the spirit and scope of the appended claims.

1. A vascular compression assist device to achieve hemostasis, comprising:
a first arm having an opening adapted for receiving one or more finger tips of a user and the first arm having a free end adjacent to the opening;
a second arm having a free end wherein the ends of the first arm and second arm are spaced apart;
a curved mid-section integrally formed with the first arm and second arm, wherein the device has a generally C-shaped configuration having a gap between the first arm and second arm for receiving a body part in which a blood vessel is disposed, and the first arm, second arm and mid-section are composed of a same shape memory material such that the first arm and second arm are self- biasing toward one another; and,
wherein the gap has a dimension from the end of the first arm to a point associated with the second arm and the dimension of the gap is less than a width dimension of a body part to be received between first arm and second arm, and the first arm and second arm bias toward one another to apply pressure to the blood vessel.

2. The device of claim 1, wherein after a body part is received within the device and between the first arm and second arm, the first arm and second arm bias toward one another and the first arm applies pressure to the blood vessel to assist in achieve hemostasis relative to the blood vessel.

3. The device of claim 2, wherein the opening enables a user to apply tactile pressure to the blood vessel to achieve hemostasis and detect hemostasis in the blood vessel by insertion of the user's finger tips through the opening of the first arm.

4. The device of claim 1, further comprising a window, at least a portion of which is disposed on the first arm and the opening of the first arm is between the window and the free end of the first arm.

5. The device of claim 3, wherein the opening of the first arm is a first opening of the device and the device further comprising a second opening in the second arm and the second opening is configured to receive at least a portion of a thumb of the user.

6. The device of claim 1, further comprising a pad on an inner surface of the first arm between an edge of the first arm and the free end of the first arm.

7. The device of claim 1, wherein the opening of the first arm includes a plurality of contiguous edges defining the opening as being generally ovular or rectangular, and the plurality of edges includes a first edge and a second edge spaced apart and parallel to the first edge, and two diametrically opposed end edges that extend radially outward and upward relative to a curvature of the device.

8. The device of claim 7, further comprising a fingertip engagement member between the second edge of the opening and the free end of the first arm.

9. The device of claim 1, wherein, the first arm, second arm and mid-section are formed as contiguous integral parts of single component device that is composed of a medical grade plastic material.

10. The device of claim 9, wherein the device is composed of a polypropylene material.

11. A method of tactile hemo stasis of a blood vessel at a surgical insertion site of the blood vessel, comprising:
providing a self-biasing C-shaped device having a first arm spaced apart from a second arm forming a gap there between for receiving a body part in which the blood vessel is disposed, and the first arm includes an opening configured to receive one or fingertips of a user of the device;
manually gripping the device by hand and opening the gap to receive the body part;
positioning the first end of the device proximal the blood vessel;
applying pressure to the blood vessel by allowing the first arm and second arm to bias toward one another;
inserting one or more fingertips through the opening;
applying pressure against the body part at the blood vessel via the fingertips through the opening to achieve and detect hemostasis of the blood vessel.

12. The method of claim 11, wherein the method is performed in relation to a surgical procedure in which a catheter is inserted into the blood vessel at the insertion site, and the step of positioning the first end proximal the blood vessel is performed before the catheter is removed from the blood pressure and the step of applying pressure via the fingertips is performed after the catheter is removed.

13. The method of claim 11, wherein the blood vessel is an artery.

14. The method of claim 12, wherein the artery is the femoral artery.

15. The method of claim 11, wherein the blood vessel is a blood vein.

16. The method of claim 11, wherein the step of inserting the one or more fingertips is performed when the device is manually gripped to open the gap.

17. A vascular compression assist device to achieve hemostasis, comprising:
a generally C-shaped self-biasing member having a first free end space apart from a second free end forming a gap there between and the first end and second end bias in directions toward one another;
an opening through the member adjacent to the first end, and the opening is configured to receive one or more fingertips of a user; and,
the member is composed of a medical grade plastic material so the member is resilient to open the gap adapting the member to receive a body part within which a surgically punctured blood vessel.

18. The device of claim 17, wherein the opening is a first opening the device further comprises a second opening through the member toward the second free end thereof for receiving a portion of a thumb of a user.

19. The device of claim 17, further comprising a window formed in the member adjacent the first opening and the first opening is between the window and the first free end of the member.

20. The device of claim 19, wherein in the member includes a solid curved section from an edge of the window to an end of the second opening.
